(19) United States
(12) Patent Application Publication
(10) Pub. No.: US 2011/0046709 A1
(43) Pub. Date: Feb. 24, 2011

(54) METHODS FOR IMPLANTING A STENT USING A GUIDE CATHETER

(75) Inventors: Lorcan J. Coffey, Tubingen (DE); Diem U. Ta, San Jose, CA (US); Randolf von Oepen, Los Altos, CA (US); Fozan O. El-Nounou, Santa Clara, CA (US); Laura M. Kalvass, Mountain View, CA (US)

Correspondence Address: FULWIDER PATTON, LLP (ABBOTT), 6060 CENTER DRIVE, 10TH FLOOR, LOS ANGELES, CA, 90045, US

(73) Assignee: ABBOTT CARDIOVASCULAR SYSTEMS, INC., Santa Clara, CA (US)

(21) Appl. No.: 12/543,340

(22) Filed: Aug. 18, 2009

(51) Int. Cl. A61F 2/84

(52) U.S. Cl. 623/1.11

(57) ABSTRACT

Various methods for implanting a stent into an ostium or a proximal portion of a body vessel utilizing a guide catheter, rather than a convention balloon angioplasty balloon catheter, as the primary delivery device are disclosed. The guide catheter can be used as the primary stent delivery catheter for the ostium region of the body vessel and can be anchored to the vessel to allow secondary treatment devices to be advanced to distal locations in the body vessel.

### BACKGROUND OF THE INVENTION

The invention relates to the field of intraluminal catheters, and particularly, to guiding catheters suitable for intravascular procedures such as angioplasty, stent deployment and the like.

In percutaneous transluminal coronary angioplasty (PTCA) procedures, a catheter system is introduced into a patient's vasculature to open up a body lumen, usually a coronary artery, which has become partially blocked by a stenotic lesion. These procedures can also be used to open up other arteries which have become partially blocked, including, but not limited to the carotid arteries and peripheral arteries. The catheter system is usually inserted into the heart to treat a coronary artery by advancing the system through the femoral artery, although it is possible to access the coronary arteries through other arteries in the patient's vasculature.

The catheter system usually includes a guide catheter, often referred to as a guiding catheter, which is used to create an open passageway for the insertion of other catheters which are to be advanced to the target lesion. The guide catheter usually has a pre-shaped distal section which allows it to be positioned in the proper direction once placed in the aortic arch of the patient. The guide catheter is usually percutaneously introduced into the patient's vasculature and then advanced through the patient's vasculature until the shaped distal section of the guide catheter is adjacent to the ostium of a desired coronary artery. The proximal end of the guiding catheter, which extends out of the patient, is torqued to rotate the shaped distal section and, as the distal section rotates, it is guided into the desired coronary ostium. The distal section of the guide catheter is shaped so as to engage a surface of the ascending aorta and thereby seat the distal end of the guide catheter in the desired coronary ostium and to hold the catheter in that position during the procedures when other intravascular devices such a guide wires, balloon catheters and stent catheters are being advanced through the inner lumen of the guiding catheter.

In the typical PTCA or stent delivery procedures, the balloon catheter with a guide wire disposed within an inner lumen of the balloon catheter is advanced within the inner lumen of the guide catheter which has been appropriately positioned with its distal tip seated within the desired coronary ostium. A guide wire is first advanced out of the distal end of the guide catheter into the patient's coronary artery until the distal end of the guide wire crosses a lesion to be dilated or an arterial location where a stent is to be deployed. A balloon catheter is advanced into the patient's coronary anatomy over the previously introduced guide wire until the balloon on the distal portion of the balloon catheter is properly positioned across the lesion. Once properly positioned, the balloon is inflated with inflation fluid one or more times to a predetermined size so that in the case of the PTCA procedure, the stenosis is compressed against the arterial wall, and the wall is expanded to open up the vascular passageway. In the case of stent deployment, the balloon is inflated to plastically expand a stent within the stenotic region where it remains in the expanded condition. Generally, the inflated diameter of the balloon is approximately the same diameter as the native diameter of the body lumen being dilated so as to complete the dilatation or stent deployment but not over expand the artery wall. After the balloon is finally deflated, blood flow resumes through the dilated artery and the dilatation catheter and the guide wire can be removed therefrom. Generally, the stent deployment may be accomplished simultaneously with or after a PTCA procedure has been performed at the stenotic site.

Treatment of the main coronary arteries (left and right) usually requires the guide catheter to be engaged within the ostium of the artery, as will be explained. Unfortunately, the anatomy of the main coronary arteries, particularly the left main coronary artery, can be relatively short and can compromise the support provided by the guiding catheter. Additionally, lesions near the ostium can prevent a deep seating of the distal end of the guide catheter within the ostium. Guide catheter backout can thus occur when an interventional device, such as a balloon catheter, is used within the distal portion of the artery. “Backout” of the guide catheter from the ostium is a term used to describe the action taken by the guide catheter caused by the reactive forces applied to the guide catheter as the interventional device is treating the lesion. As the term implies, the guide catheter will move or back out of the ostium, which is detrimental to its function. As a result, it can be difficult to place a stent in the region where the distal end of the guide catheter is seated, and if the guide catheter is not properly seated, it can back out.

What has been needed is a guide catheter design which would allow the implantations of a stent in the ostium or proximal region of the coronary artery while not compromising the support that is need from the guide catheter for accurate placement of the primary or secondary stents or other treatment devices. The present invention satisfies these and other needs.

### SUMMARY OF THE INVENTION

The present invention is generally directed to methods for implanting a stent into an ostium or a proximal portion of a body vessel utilizing a guide catheter, rather than a conventional balloon angioplasty catheter, as the primary delivery device. By using the guide catheter as the primary stent delivery catheter, the guide catheter can function both as a conventional guide catheter to allow interventional instruments to be advanced therethrough and as a stent delivery catheter to simultaneously implant a stent in a location of the body vessel which is often difficult to implant a stent with conventional stent delivery devices. The present invention is particularly useful in allowing a physician to accurately implant a stent near the ostium of a main coronary artery branching from the aorta, while still achieving adequate guide catheter support for advancement of secondary treatment devices into the main coronary artery.

The methods of the present invention can utilize a guide catheter which includes a mounting region located at its distal end section upon which a stent can be mounted. Suitable stents which can be placed on the mounting region include, but are not limited to, balloon-expandable stents, self-expanding stent, bio-absorbable stents, drug-eluting stents and composite stents. When a balloon expandable stent is to be implanted, the guide catheter should include an expandable member, such as a balloon, which is adapted to apply an outward radial force which expands the stent into the body lumen. The guide catheter would require a mechanism, such as an inflation lumen and an inflation port, which would be used to inflate the balloon. When a self-expanding stent is to be implanted, usually a retractable sheath would be co-axially disposed over the guide catheter to maintain the self-expanding stent in a collapsed, delivery position. The retractable sheath could be simply retracted to remove the restraint placed on the self-expanding stent, thus allowing the self-expanding stent to radially expand to its expanded position. A retractable sheath also could be used to cover a balloon-expandable, bio-absorbable and drug eluting stents to protect these stents as the guide catheter is being advanced into the patient's vasculature. As a result of using a protective outer sheath, there may be no need to crimp the stent onto the guide catheter, which would be beneficial especially if the stent has a fragile structure or is coated with a drug. The guide catheter can be made similar to conventional devices and include an elongated shaft with a preshaped or shapeable distal shaft section to facilitate placement of the distal tip of the guide catheter into a patient's body lumen.

A method of implanting a stent into a patient's vasculature utilizing a guide catheter includes providing a guide catheter having a distal section, a proximal section and an inner lumen adapted to receive interventional devices therethrough. The distal section of the guide catheter has a stent disposed thereon. The guide catheter and stent are then advanced through the patient's vasculature to a location where a branch vessel, such as the left main coronary vessel, forms from a main vessel, such as the aorta. The distal section of the guide catheter and the stent can then be positioned into the opening, or ostium, of the branch vessel a sufficient distance to anchor the distal section of the guide catheter within the branch vessel. The method further includes implanting the stent at the opening of the branch vessel to treat a stenosis which has formed at or in close proximity of the ostium of the branch vessel.

In a further method of use, an interventional device can be advanced through the lumen of the guide catheter after the stent is implanted. As mentioned above, the guide catheter may include an expandable member upon which the stent is mounted. In using such a guide catheter, the method can include actuating the expandable member to implant the stent in the opening of the branch vessel, which will also help in anchoring the distal section of the guide catheter within the branch vessel. The expandable member can be, for example, an inflatable stent balloon. After the expandable member has been actuated, an additional interventional procedure(s) can be performed on one or more stenoses, lesions or damaged vessel located distal to the ostium by simply using the guide catheter to advance the necessary interventional devices through the lumen of the guide catheter. As a result of this method, the physician does not have to utilize a conventional stent delivery catheter to stent the sometimes difficult to stent ostium of the branch vessel.

As mentioned above, the stent can be covered during the time that the guide catheter is being advanced through the patient's vasculature. The method contemplates retracting the sheath in order to implant the stent. When the stent is a self-expanding type, the simple retraction of the sheath should allow the stent to self-expand to its expanded position. If the stent is a balloon expandable type, the sheath should first be retracted, otherwise the expansion of the balloon could cause the sheath to expand with the stent. Thereafter, the expandable member can be actuated to implant the stent at the area of stenosis in the ostium of the branch vessel. In one particular method of the present invention, the sheath can be made from a bio-absorbable material which starts to dissolve once making contact with the body fluid. In such a method, the sheath does not have to be retracted since the sheath will dissolve in time to expose the stent for implantation.

The guide catheter can be made from suitable polymeric material and may include multistrand reinforcing structure made from metallic or metallic like materials which extend through at least a portion of the shaft section of the guide catheter. The distal section of the guide catheter is usually more flexible to provide atraumatic characteristics to the distal tip. The distal end of the catheter shaft is typically provided with an atraumatic distal tip which can be radiopaque to enhance visibility under a fluoroscope. Additionally, the distal section of the guide catheter can be pre-shaped, common with conventional guide catheters, in order to match the vasculature in which the guide catheter is to be placed. Suitable polymeric materials include, but are not limited to polyimides, polyamide elastomers, e.g., polyether block amides such as PEBAX alone or blended with nylon or PEBAX materials with other durometers. Other suitable polymeric materials include polyurethanes. A variety of other thermoplastic and thermoelastic polymers, copolymers and blends may also be employed.

These and other advantages of the invention will become more apparent from the following detailed description of the invention and the accompanying exemplary drawings.

### BRIEF DESCRIPTION OF THE DRAWINGS

FIG. 1 is a side view of a guide catheter and protective outer sheath which can be used in conjunction with the method of the present invention showing the distal section of the guide catheter being inserted into the ostium of the left main coronary artery;

FIG. 2 is a side view of the guide catheter of FIG. 1 with the protective outer sheath being retracted to expose a stent within the ostium of the left main coronary artery;

FIG. 3 is a side view of the guide catheter of FIG. 1 with the stent being expanded by an expandable member, such as an inflatable balloon, to implant the stent within the ostium of the coronary artery and to anchor the guide wire within the vessel to allow another interventional procedure to be performed in the left distal main coronary artery;

FIG. 4 is a side view showing the stent which has been implanted within the ostium of the left main coronary artery and a stent which has been implanted in the left main coronary artery;

FIG. 5 is a side elevational view of a guide catheter invention;

FIG. 6 is a side view of a guide catheter which can be used in conjunction with the method of the present invention that includes a retractable outer sheath and a mechanism for moving the sheath;

FIG. 7 is an embodiment of an outer sheath which protects the stent mounted on the guide catheter and is dissolvable when subjected to blood or other bodily fluids;

FIG. 8 is a side prospective view showing a guide catheter being inserted into an introducer assembly which has been placed into the vasculature of a patient; and

FIG. 9 is another embodiment of a splitable outer sheath which protects the mounted stent and can be easily retracted from the guide catheter utilizing simple proximal retraction which causes the sheath to split from the guide catheter.

### DETAILED DESCRIPTION OF THE PREFERRED EMBODIMENTS

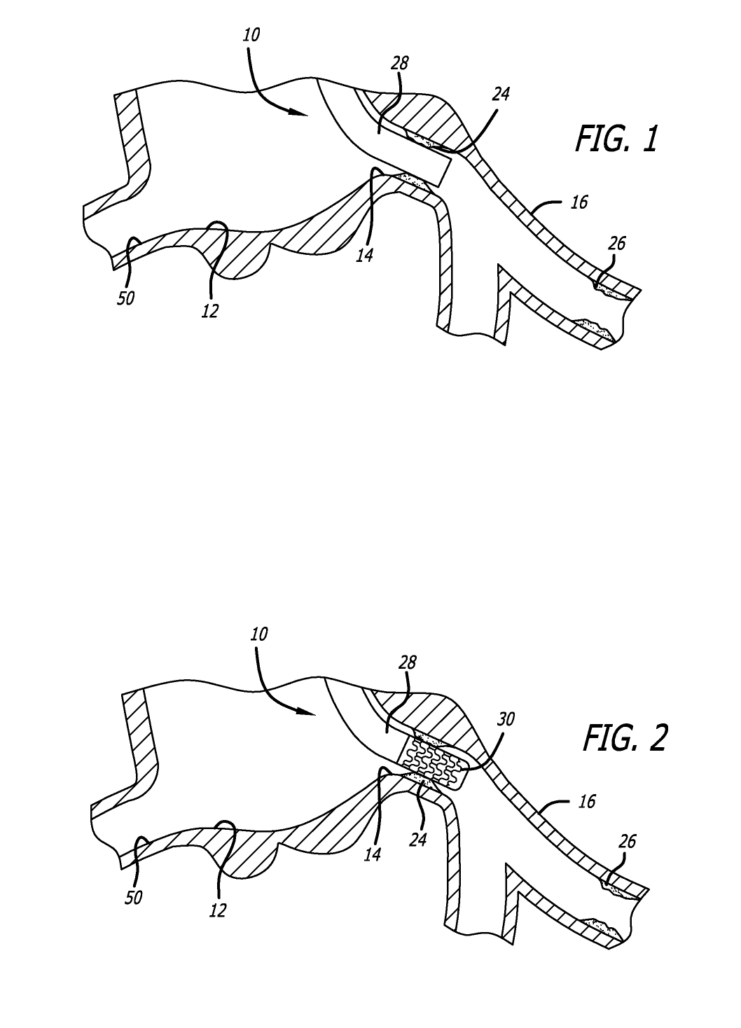

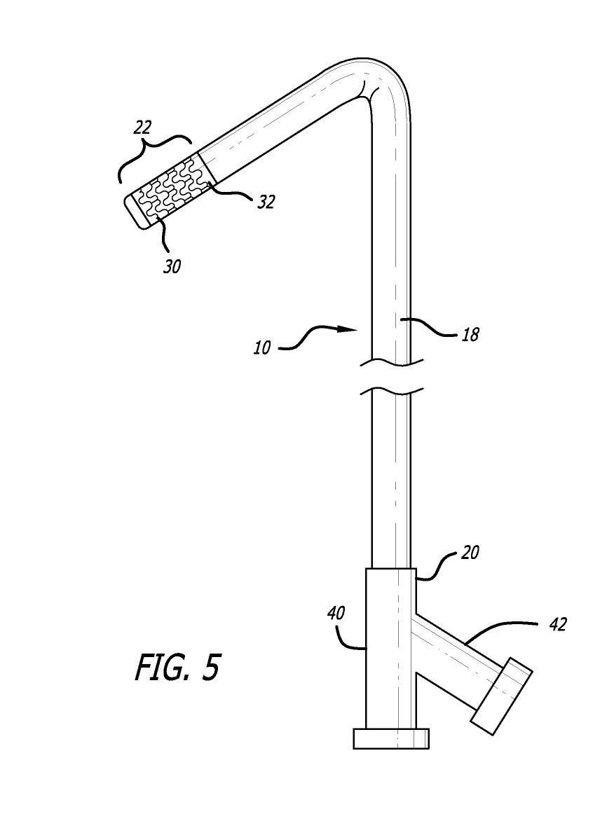

Referring initially to FIGS. 1-4, a guide catheter 10 is depicted as it is being place in a patient's vasculature to implant a stent into an ostium of a body vessel extending from a main body vessel. For example, the guide catheter 10 can be advanced through the femoral artery to the aorta 12 for implanting a stent in the opening, or ostium 14, of the left main coronary artery 16. FIG. 5 illustrates one specific embodiment of a guide catheter 10 which can be used to implant the stent using the methods of the present invention. FIG. 5 shows the guide catheter 10 including a generally elongated catheter shaft 18 with a proximal section 20 and a distal section 22. In FIGS. 1-3, only the distal section 22 of the guide catheter 10 is shown being placed in the patient's vasculature. It should be appreciated that the proximal section 20 extends out of the patient to allow the physician to advance conventional interventional instruments into the left main coronary artery 16.

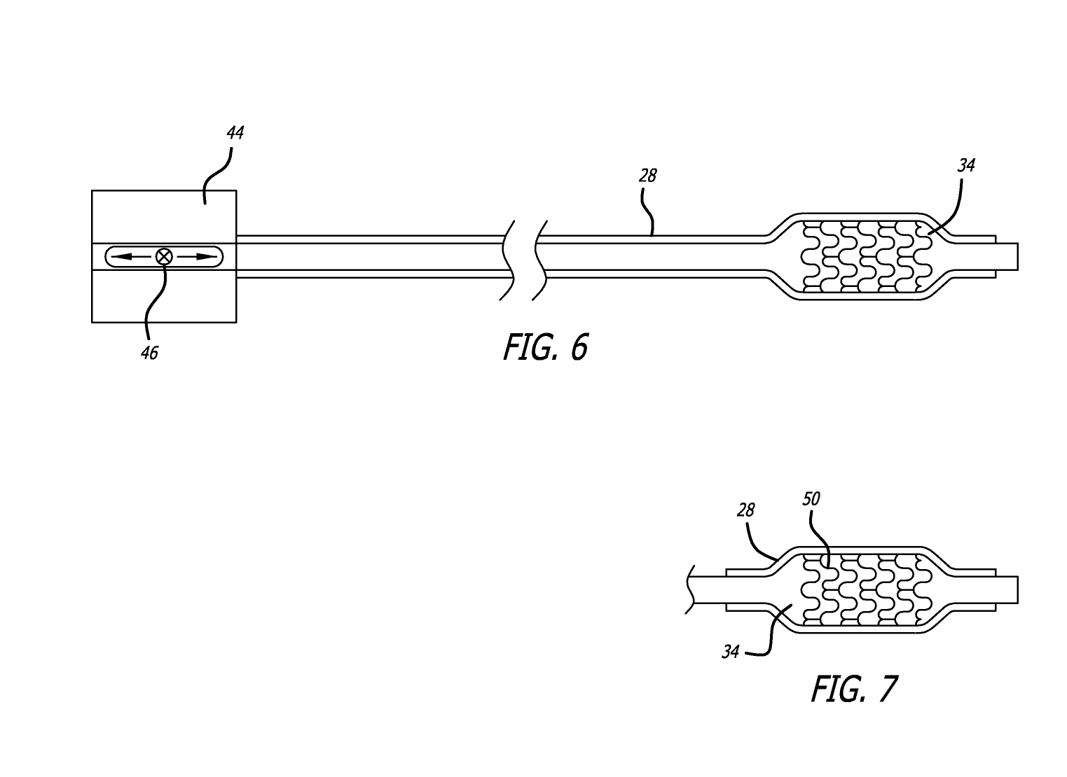

The distal section 22 of the guide catheter 10 is shown in FIG. 1 initially being inserted into the ostium 14 of the left main coronary artery 16. As can be seen in FIGS. 1 and 2, a lesion or stenosis 24 is located at or near the ostium 14 and is in need of treatment. Another lesion or stenosis 26, located further within the left main coronary artery 16, requires treatment as well. In the particular sequences of steps being performed in FIGS. 1-4, a retractable sheath 28 (See FIG. 6) is initially shown as it extends co-axially over the guide catheter 10 to protect the stent 30 which is mounted in a stent mounting region 32 located on the outer surface of the guide catheter 10 (see FIGS. 5 and 6). The stent mounting region 32 may be formed from an expandable member, such as an inflatable balloon 34, which can be used to expand the stent 30 within the ostium 14 of the artery 16.

As is shown in FIG. 1, the distal section 22 of the guide catheter 10 is initially covered by the sheath 28 to protect the stent 30 as the guide catheter 10 is being advanced within the patient's vasculature. This sheath 28 also helps to prevent the stent 30 from scraping the walls of the patient's vasculature. Once the distal section 22 is placed into the ostium 14, in contact with the stenosis, the sheath 28 can then be retracted to expose the stent 30, as is shown in FIG. 2. Alternatively, the distal section 22 of the guide catheter 10 can be brought close to the ostium 14 of the left main coronary artery 16. Then, the sheath 28 can be removed from the stent 30 to allow the physician to advance the distal section 22 of the guide catheter 10 and the stent 30 into the ostium 14.

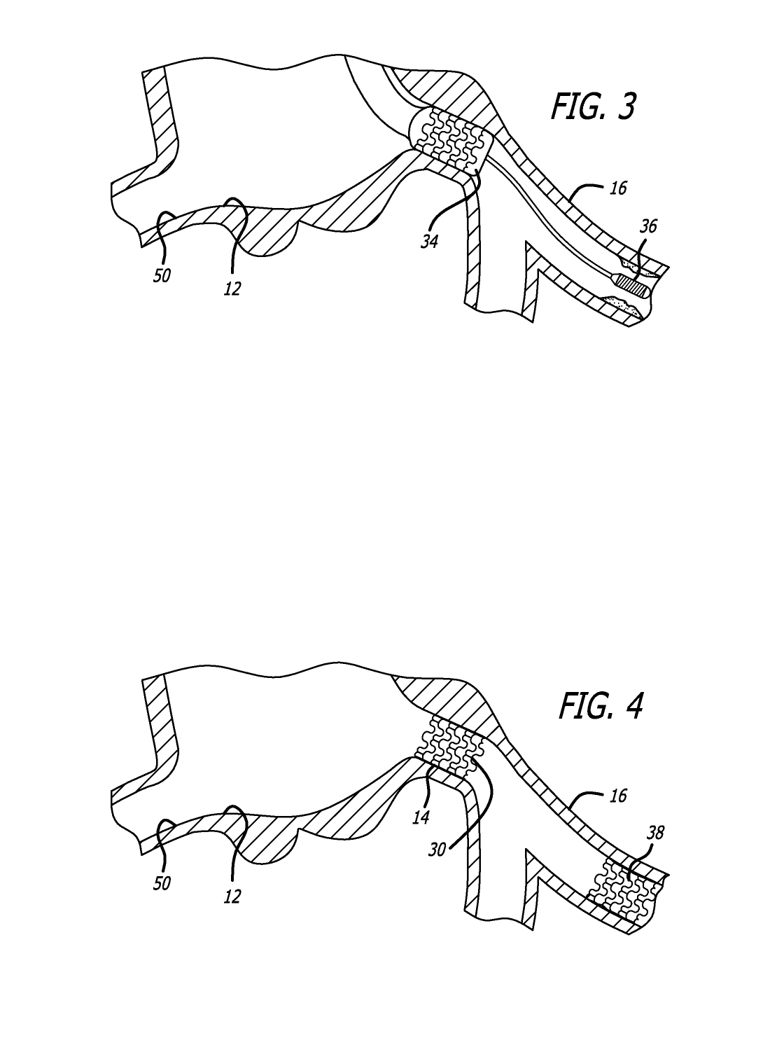

Next, as is depicted in FIG. 3, the expandable member 34 of the guide catheter 10, namely, the inflatable balloon 34, can be expanded in order to radially expand the stent 30 in the ostium 14 to treat the stenosis 24. The expansion of the inflatable balloon 34 also causes the distal section 22 of the guide catheter 10 to be anchored within the ostium 14, providing a solid “anchoring” of the guide catheter to the artery 16. This allows the guide catheter 10 to be used to advance interventional devices through its inner lumen (not shown) so that additional procedures can be performed within the left main coronary artery 16. As can be seen in FIG. 3, a conventional guide wire 36 can be advanced through the inner lumen (not shown) of the guide catheter 10 to the target location where the second stenosis 26 is located. This guide wire 36 can be used to advance conventional interventional instruments, such a stent delivery catheter (not shown) into the target location. It should be appreciated that stenoses are shown in the disclosed method of using the guide catheter to implant a stent at the ostium. It should be appreciated that the stenosis could also be any other type of damage to the artery, such as a detached lining. Also, while a stenting procedure is shown in this particular disclosed method, it should be appreciated that other interventional procedures could be performed, such as a simple angioplasty procedure, an ablation procedure and the like. Multiple procedures could be performed as well. For example, multiple stents could be implanted in the artery 16 when multiple stenoses are present.

FIG. 4 shows the left main coronary artery 16 after the guide catheter 10 has been removed from the vasculature. As can be seen, a stent 30 is implanted at the ostium 14 with an additional stent 38 implanted distally within the artery 16. As a result, the stenoses 24 and 26 have been properly treated.

The above-described method utilizes a balloon expandable stent to be implanted at the ostium of the branch vessel. Other stents can be implanted at the ostium as well. For example, a self-expanding stent can be implanted. A protective sheath is used when implanting a self-expanding stent in order to maintain the stent in a collapsed, delivery position. The sheath and self-expanding stent must first be placed into the ostium 14, as is shown in FIG. 1. Once in place, the sheath 28 can be retracted, as is shown in FIG. 2, to allow the stent 30 to radially expand. While a self-expanding stent does not need the inflatable balloon 34 in order to expand, a physician might still inflate the balloon to ensure that the self-expanding stent is fully apposed to the arterial wall. The inflation of the balloon also can assist in compressing the plaque that forms the stenosis 24. A retractable sheath 28 is also helpful when a fragile bio-absorbable or drug eluting stent is to be implanted in the ostium. The sheath 28 can protect these types of stents as the guide catheter 10 is being advanced into the patient's vasculature. Additionally, by using a retractable sheath, there may be no need to crimp the stent onto the guide catheter, which would be beneficial especially when the stent has a fragile structure (bio-absorbable) or is coated with a drug.

Referring again to FIGS. 5 and 6, the proximal section 20 of the guide catheter 10 may include a fitting which includes an inflation port 42 used to inject inflation fluid into an inflation lumen (not shown) that is in fluid communication with the inflation balloon 34. The elongate shaft 18 of the guide catheter 10 would include a main lumen (not shown) through which the interventional instruments are advanced through during usage. These lumens of the shaft 18 can be formed in any one of a number of configurations well known in the catheter art. Referring specifically to FIG. 6, the sheath 28 that extends over the guide catheter 10 may include a deployment mechanism 44 which can be used to move the sheath proximally. A control knob 46 can be easily manipulated to retract the sheath 28 axially along the length of the guide catheter 10. Such deployment mechanisms 44 are well known in the art and can be manufactured in any one of a number of ways.

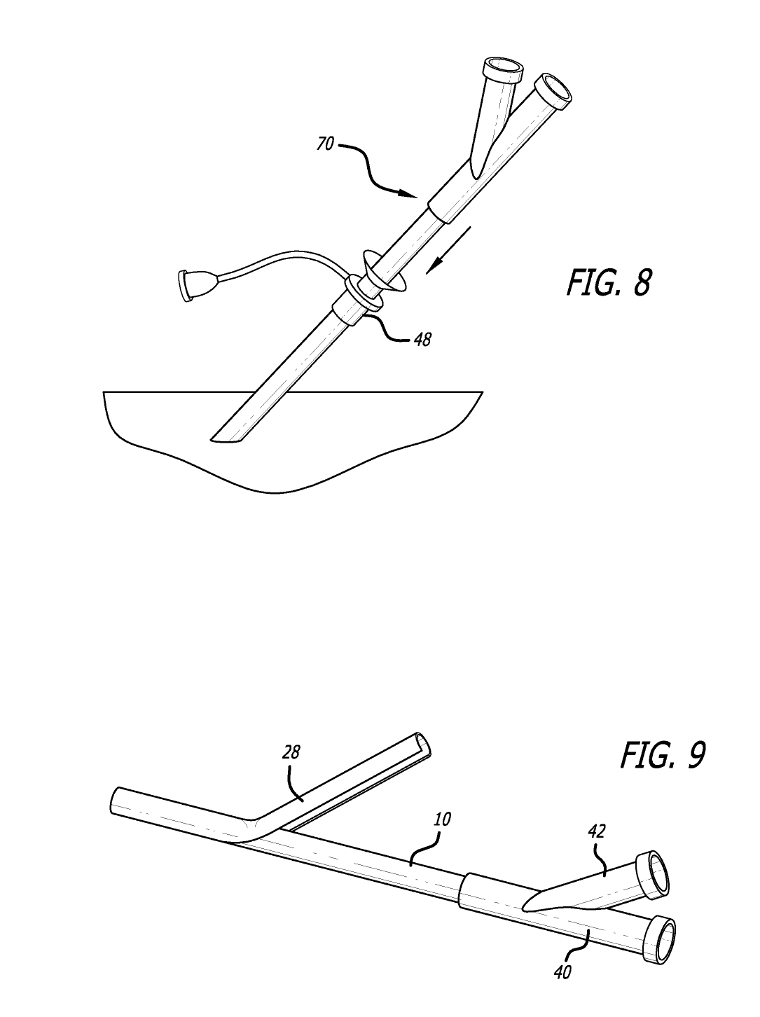

FIG. 7 shows another way in which to protect the stent 30 as it is being advanced through the patient's vasculature. In this particular method, the sheath 28 can be made from a bio-absorbable material, such as polysaccharide, which starts to dissolve once the sheath 28 makes contact with the body fluid in the vessel. In such a method, the sheath 28 does not have to be retracted since the sheath 28 will eventually dissolve in time to expose the stent 30 for implantation. FIG. 8 shows the use of an introducer 48 which is typically used in an interventional procedure. The introducer 48 is initially inserted into the patient's vasculature using a Seldinger technique. However, when the guide catheter is being advanced within the lumen of the introducer 48, it is possible for an uncovered stent to be accidentally bumped or moved from its mounted position on the stent mounting region. Accordingly, the use of the protective retractable sheath 28 will help to prevent this from occurring. FIG. 9 shows yet another embodiment of a retractable sheath 28 which can be easily removed from the guide catheter during use. For example, such a sheath can utilize a structure which allows the wall of the sheath to be split as the sheath is being removed from the guide catheter. For example, a portion of the wall could be made thinner than the remaining portion of the wall. The thinner portion of the wall will be more susceptible to shearing. Alternatively, a perforation or pre-formed split can be made along the length of the tubing forming the sheath to permit the sheath to split once the physician starts to retract the sheath from the guide catheter. It should be appreciated that such a splitable sheath will provide a rapid means for completely removing the sheath from the guide catheter. However, in use, the sheath only has to be retracted a short length to expose the stent mounted on the guide catheter.

While the disclosed method describes the use of the guide catheter in implanting a stent into the ostium of the left main coronary artery, it should be appreciated that the method of the present invention could be used in a number of branch vessels which are susceptible to lesions and stenoses near the ostium of the vessel. For example, the guide catheter could be used to implant a stent at the right main coronary artery 50, shown in FIGS. 1-4. Additionally, the distal section of the guide catheter can include a pre-formed shape which allows the guide catheter to be easily positioned in the desired branch vessel.

The guide catheter can be made from materials used in conventional guide catheters and balloon dilatation delivery systems. For example, the guide catheter could be made from a polymeric material and may include a reinforcing structure or layer formed from stiffer materials, such as braiding or winding formed from wires. Such construction for a guide catheter is well known in the art. It is contemplated that the reinforcing structure could include strands of wires formed of stainless steel and similar materials. Most of the strands forming the braided reinforcing structure are formed of a high strength, highly radiopaque metal. A substantial portion of the strands can be formed of a variety of other materials that include stainless steel (304) and high strength alloys such as MP35N, Elgiloy, Conichrome, Haynes 242 and L-605 which contain cobalt, chromium and nickel as well as high strength polymeric material. High strength plastic strands (e.g., Kevlar) or mixtures of plastic and metallic strands may also be used to form part of the reinforcing structure of the guide catheter.

Guide catheters designed for coronary artery access have varying lengths, generally between about 90 to about 130 cm, in one embodiment the length is about 100 to about 120 cm. The wall thickness of the catheter shaft can ranges from about 0.003 to about 0.01 inch (0.076-0.254 mm). The guide catheter can have a generally constant outer diameter throughout its length, and the wall thickness can also be generally constant.

It will be apparent from the foregoing that, while particular methods of the invention have been illustrated and described, various modifications can be made without departing from the spirit and scope of the invention. While the description of the invention is directed to embodiments for coronary applications, various modifications and improvements can be made to the invention without departing therefrom.

We claim:

1. A method of implanting a stent into a patient's vasculature utilizing a guide catheter, comprising:
providing a guide catheter having a distal section, a proximal section and an inner lumen adapted to receive interventional devices therethrough, the distal section of the guide catheter having a stent disposed thereon;
advancing the guide catheter and stent through the patient's vasculature to a location where a branch vessel forms from a main vessel;
positioning the distal section of the guide catheter and the stent into the opening of the branch vessel a sufficient distance to anchor the distal section of the guide catheter within the branch vessel; and
implanting the stent at the opening of the branch vessel.

2. The method of claim 1, wherein the method further includes:
advancing an interventional device through the lumen of the guide catheter and performing an interventional procedure in a region distal to the opening of the branch vessel.

3. The method of claim 2, wherein the interventional device is advanced through the lumen of the guide catheter after the stent is implanted.

4. The method of claim 1, further including a retractable sheath disposed over the stent as the guide catheter is advanced through the patient's vasculature.

5. The method of claim 4, wherein the method further includes retracting the sheath to implant the stent.

6. The method of claim 5, wherein the guide catheter includes an expandable member upon which the stent is mounted and the method further includes retracting the sheath to expose the stent and actuating the expandable member to expand the stent.

7. The method of claim 5, wherein the stent is a self-expanding stent.

8. The method of claim 1, wherein the guide catheter includes an expandable member upon which the stent is mounted and the method further includes actuating the expandable member to implant the stent and to anchor the distal section of the guide catheter in the branch vessel.

9. The method of claim 8, wherein the method further includes maintaining the expandable member expanded in the opening of the body vessel and advancing an interventional device through the lumen of the guide catheter and performing an interventional procedure in a region distal to the opening of the branch vessel.

10. The method of claim 9, wherein the interventional procedure is a stenting procedure.

11. The method of claim 4, wherein the sheath is made from a bio-absorbable material which starts to dissolve when contacting body fluid.

12. A method of implanting a stent by utilizing a guide catheter into a main coronary vessel that branches from the aorta of a patient, comprising:
providing a guide catheter having a distal section, a proximal section and an inner lumen adapted to receive interventional devices therethrough, the distal section of the guide catheter having an expandable member upon which a stent is disposed;
advancing the guide catheter and stent through the patient's vasculature into the aorta and near the desired main coronary vessel;
positioning the distal section of the guide catheter and the stent into the opening of the main coronary vessel a sufficient distance; and
actuating the expandable member to implant the stent at the opening of the main coronary vessel and to anchor the distal section of the guide catheter to the main coronary vessel.

13. The method of claim 12, wherein the method further includes:
advancing an interventional device through the lumen of the guide catheter and performing an interventional procedure in a region distal to the opening of the main coronary vessel.

14. The method of claim 13, wherein the interventional device is advanced through the lumen of the guide catheter as the expandable member remains actuated.

15. The method of claim 12, further including a retractable sheath disposed over the stent and wherein the method includes retracting the sheath prior to actuating the expandable member.

16. The method of claim 15, wherein the guide catheter has a pre-formed shape associated with the distal portion.

17. The method of claim 13, wherein the interventional procedure is a stenting procedure.

18. The method of claim 12, wherein the expandable member is an inflatable balloon.

19. The method of claim 13, wherein the sheath is made from a bio-absorbable material which starts to dissolve when contacting body fluid.

20. The method of claim 12, wherein the main coronary vessel is the left main coronary vessel.
